(19) United States
(12) Patent Application Publication
(10) Pub. No.: US 2023/0241134 A1
(43) Pub. Date: Aug. 3, 2023

(54) PROBIOTIC COMPOSITION FOR TOPICAL USE

(71) Applicant: BIOGAIA AB, Stockholm (SE)

(72) Inventors: Christoffer LUNDQVIST, Lund (SE); Eile BUTLER, Cork (IE); Jonas FAIJERSON SÄLJÖ, Göteborg (SE)

(21) Appl. No.: 18/002,670

(22) PCT Filed: Jun. 30, 2021
(86) PCT No.: PCT/EP2021/068084

§ 371 Date: Dec. 21, 2022

(30) Foreign Application Priority Data
Jun. 30, 2020 (SE) 2050790-1

(51) Int. Cl. A61K 35/747; A61K 47/44; A61J 1/14; A61K 9/06; A61K 8/99; A61K 8/92; A61K 9/00; A61P 17/06; A61Q 19/00

(52) U.S. Cl. CPC A61K 35/747; A61K 47/44; A61J 1/1468; A61K 9/06; A61K 8/99; A61K 8/922; A61K 9/0014; A61P 17/06; A61Q 19/007

(57) ABSTRACT

The present invention relates to the field of topical compositions comprising probiotic bacteria showing improved properties for storage and stability. More particularly, the invention relates to such topical compositions for cosmetic and medical use and to containers and kits comprising such compositions. Storing the compositions in containers together with a solid support comprising a desiccant polymer was proven to provide excellent storage prerequisites to the composition comprising the probiotic bacteria.

### FIELD OF THE INVENTION

The present invention relates to topical probiotic products, more specifically to topical ointment compositions comprising probiotic bacteria for cosmetic or medical use. The present invention also relates to the packaging, storing and long-term stability of such ointment compositions together with a moisture absorbing material, said compositions being sensitive to moisture due to the presence of viable probiotic bacteria therein.

### BACKGROUND OF THE INVENTION

The Food and Agricultural Organization of the United Nations has defined probiotics as “live microorganisms which, when administered in adequate amounts, confer a health benefit on the host”. Several different bacterial strains are currently used as probiotics, including lactic acid producing bacteria, such as strains of Lactobacillus and Bifidobacteria. The effectiveness of probiotic bacteria is strain-specific, and each strain may contribute to host health through different mechanisms.

A general problem encountered in dried microorganism preparations (e.g. freeze-dried) is the limited storage time of the cells since even minute amounts of free water content is deleterious for these moisture sensitive cells. Over time the microorganisms therefore become less viable resulting in e.g. a requirement for higher dosages to compensate for the loss of activity or in problems generating desired product formulations for the intended use. The use of preservatives in these formulations is furthermore not recommended due to the antimicrobial effect they would have against the added microorganism in the formulation.

One solution to maintain a particularly low level of moisture within a package is to incorporate sachets of desiccant material into the internal space of the package to remove the moisture from the air in the headspace of the package. The desiccant material is generally known to reduce the moisture content within a package. Typical desiccant materials are “physical” desiccant materials, such as molecular sieves that bind water molecules within pore spaces of a material. Another type of desiccant material includes hydrate forming agents such as salts, such as ammonium chloride.

Desiccant materials may also be used that form no hydrates, such as common salt (NaCl) or potassium bromide (KBr).

One way to protect freeze-dried and other types of dried lactic acid bacteria cultures from moisture is to use an oil-based formulation. Lactic acid bacteria cells have previously been used in oil formulations for improved stability of the bacteria, see for example U.S. Pat. No. 4,518,696. In U.S. patent application publication No. 20050271641, a step of vacuum-drying the oil was performed before formulation to achieve increased stability of the bacterial cultures and a product called “Reuteri Drops” was manufactured. This product is an oil-based formulation containing L. reuteri made for good stability and shelf life. The unique feature of this production process is the drying step of the oil to remove most of the water.

WO2012038414 discloses a method for the storage of freeze-dried lactic acid bacteria by combining a non-water liquid, i.e. a liquid oil with a moisture absorbing polymeric material or a multilayer plastic polymeric flexible packaging foil having a chemical desiccant material incorporated within a layer of foil. The material used was efficient in absorbing moisture from the oil (i.e. a liquid composition) and was thereby capable of improving the stability and storage of the lactic acid bacteria present therein.

In recent years probiotics and their derivatives have begun to be investigated also for topical use in terms of safety and efficacy on skin. Investigations have for example been made in subjects with sensitive or reactive skin, atopic dermatitis or acne prone skin. Multiple bacterial strains have been investigated and have been shown to have anti-inflammatory, antimicrobial and barrier function properties which have benefits in different skin conditions. However, the same problems with limited storage time and viability exist for topical probiotic products as for other probiotic products. This is due to the fact that they contain live bacteria and as a result of this most topical products containing bacteria have used bacterial lysates instead of live bacteria. There is also a need to provide topical products comprising probiotics that have a texture that is comfortable for the user, lacking eventual graininess and lumps that may occur in such products. Products containing probiotics are also sensitive to changes in color and the development of unpleasant smells during storage and long-term use.

As mentioned, topical probiotic products as such are available in the art. Such products have been described both for cosmetics as well as for medical purposes. As an example, EP2364712A1 discloses a cosmetic or pharmaceutical product for topical application comprising a primary oily phase and a complementary aqueous phase, said primary oily phase comprising an effective amount of at least one viable probiotic bacteria strain.

There is a need for new and improved topical probiotic products containing bacteria for treating skin conditions or improving skin in general, both for medical and cosmetic purposes. In this regard, a problem exists with the storage and stability of such probiotic products as bacteria are sensitive to moisture and oxidation. This is also highly relevant for probiotic products for topical use. Hence, there is a need to identify topical probiotic products that are improved for storage and stability in addition to be being beneficial for the user for medical or cosmetic purposes.

### SUMMARY OF THE INVENTION

The above presented problems have now thus been overcome, or at least mitigated, by the provision herein of improved ointment compositions comprising a fine grain culture powder of dried (e.g. freeze-dried) viable probiotic bacteria for medical and cosmetic uses as well as containers and kits comprising such ointment compositions that for improved storage are provided together with a desiccant polymer present on a solid support. The ointment compositions are topical products, i.e. products for topical application to the skin of a user (subject), such as a human or an animal (such as a mammal).

In this regard it was surprisingly shown herein that the desiccant polymer present on a solid support was able to absorb a significant amount of moisture from the ointment composition, said ointment composition comprising the fine grain culture powder of probiotic bacteria and a semi-solid or solid anhydrous base formulation when the desiccant polymer is stored in combination with the ointment composition. Thereby, the moisture content in the ointment composition remained low providing for improved long-term stability of the composition and viability of the probiotic bacteria present therein. The desiccant polymer is stored in direct contact with the ointment formulation, as further described herein.

It was shown herein that when stored together with the desiccant polymer, the viability of the probiotic bacteria present as a fine grain culture powder in the ointment composition was at least the same as when a standard grain culture powder of probiotic bacteria was used in the same ointment composition together with a desiccant. This is surprising for the reasons presented further herein.

The desiccant polymer was not expected to provide the herein shown moisture-absorbing properties when used in combination with a solid or semi-solid phase anhydrous base formulation forming part of the ointment composition. One reason is that desiccant polymers are usually presented together with liquids or gas phases for this purpose and are not expected to work on solid or semi-solid phase formulations. The effect of the desiccant polymer on the viability of the fine grain culture powder of dried probiotic bacteria was even more surprising as a fine grain culture powder is more exposed to moisture than the standard grain culture powder due to the increased surface area of the fine grain culture powder. In all, this indicates that the combination of the ointment composition comprising a solid or semi-solid anhydrous formulation and the fine grain culture powder of probiotic bacteria with the desiccant polymer provides particularly beneficial characteristics that are useful for long-term storage.

Accordingly, a container comprising the ointment composition and a desiccant polymer present on a solid support was herein shown to provide a particularly suitable means for storage, stability and viability of fine grain probiotic bacteria present in an ointment composition for subsequent topical use.

It was also surprisingly shown that the ointment composition comprising the fine grain culture powder stored in this manner experienced less changes in color and smell also compared to a standard grain culture powder. The texture of the composition comprising the fine grain culture powder was also more suitable for a topical product as compared to an ointment composition comprising a standard grain culture powder. In addition to that, it also had beneficial cosmetic and/or medical effects on the user.

In summary, this is the first time that an ointment composition comprising probiotic bacteria, for uses such as presented herein, has been able to present such a broad scope of superior and surprising properties. According to the inventors' best knowledge, there are no ointment products (e.g. lotions, creams or the like) comprising probiotics on the market today that are suitable for long-term storage, that are less prone to develop odor or change in color and that provide a smooth texture that is comfortable for the user.

Accordingly, there is provided herein a container comprising: i) an ointment composition comprising an anhydrous base formulation and a fine grain culture powder of dried viable probiotic bacteria having a particle size distribution of d(90)<250 μm, wherein said anhydrous base formulation comprises a vegetable fat, a vegetable oil and a hydrogenated vegetable oil; and ii) a solid support comprising a desiccant polymer, wherein said anhydrous base formulation is a solid or semi-solid phase formulation at about 25° C.

There is also provided herein an ointment composition comprising an anhydrous base formulation and a fine grain culture powder of dried viable probiotic bacteria with a particle size distribution of d(90)<250 μm, wherein said anhydrous base formulation comprises a vegetable fat, a vegetable oil and a hydrogenated vegetable oil and wherein said anhydrous base formulation is a solid or semi-solid phase formulation at about 25° C.

There is also provided the use of such an ointment composition for non-therapeutic topical administration to a skin surface of a subject.

There is also provided herein such an ointment composition, for use in therapeutic topical administration to a skin surface of a subject.

In some aspects topical administration also includes vaginal application of said ointment composition. It may also in other aspects include application on the breast, particularly on the breast of a breast-feeding mammal, such as breast-feeding human.

There is also provided an ointment composition as disclosed herein for use in a method of treating or preventing atopic dermatitis.

There is also provided herein a method for therapeutic topical administration of an ointment composition as disclosed herein, said method comprising topically applying or administering a pharmaceutically or therapeutically effective amount of an ointment composition as disclosed herein to a skin or a mucosal surface of a subject in need thereof.

There is also provided a method for treating or preventing skin conditions or disorders, such as atopic dermatitis, in an individual, said method comprising topically, such as to a skin or a mucosal surface, applying or administering a pharmaceutically or therapeutically effective amount of an ointment composition to a subject in need thereof.

There is furthermore provided herein the use of an ointment composition as disclosed herein as a cosmetic, optionally wherein said composition is provided together with a solid support comprising a desiccant polymer, optionally wherein said solid support is selected from the group consisting of a strip, a pad, a bead or a film, or is present as a moulding in a packaging material of a container holding said ointment composition.

There is also provided herein a method for preparing an ointment composition as presented herein, said method comprising the steps of: i) mixing an anhydrous base formulation comprising a vegetable oil, a vegetable fat and a hydrogenated vegetable oil at about 65 to 70° C. until the fats are melted; ii) cooling the mixture of step i) to about 30°; and iii) mixing the anhydrous base formulation with a culture powder of dried viable probiotic bacteria to obtain an ointment composition. There is also provided an ointment composition obtainable by such a method.

In addition, there is provided by the present disclosure, a method for prolonging the shelf life and/or improving the stability of an ointment composition as disclosed herein comprising performing a method for preparing an ointment composition as previously disclosed herein, and thereafter: iv) obtaining the ointment composition of step iii) of said previously mentioned method of preparing an ointment composition and thereafter placing said composition in an air-tight container; v) placing a solid support comprising a desiccant polymer in said container together with said ointment composition, optionally wherein said solid support is a strip, a pad, a bead or a film and/or wherein said solid support comprising a desiccant polymer is present in the packaging material of said container; and vi) sealing said container. The moisture absorbing desiccant polymer is used in the packaging in such a way that it is in direct contact with the ointment composition containing the moisture-sensitive bacteria.

There is furthermore provided herein an air-tight container comprising an ointment composition and a desiccant polymer present on a solid support obtainable by said method for prolonging the shelf life and/or improving the stability of an ointment composition as disclosed herein.

In addition, there is provided the use of a solid support comprising a desiccant polymer for improving the stability and/or shelf life of an ointment composition as disclosed herein.

There is also provided the use of a solid support comprising a desiccant polymer for preventing changes in the color and/or smell of an ointment composition as disclosed herein.

### BRIEF DESCRIPTION OF THE FIGURES

FIG. 1 shows the effect of different grinding on viability of fine grain and standard Lactobacillus reuteri DSM 17938 culture powder in a topical ointment composition with desiccant (a) and without desiccant (b) over 1 month, at 37° C. Results are presented as CFU/g (or Log10 as CFU/g).

FIG. 2 shows the change in water content (ppm) in a topical ointment composition comprising fine grain or standard grain L. reuteri culture powder with desiccant (a) and without desiccant (b) over 1 month when stored at 37° C.

FIG. 3 shows the change in odor in a topical ointment composition comprising fine grain or standard grain L. reuteri culture powder with desiccant (a) and without desiccant (b) over 3 months when stored at 37° C. A score of 1 is no change while 10 is highest change in odor.

FIG. 4 shows the change in color in a topical ointment composition comprising fine grain or standard grain L. reuteri culture powder with desiccant (a) and without desiccant (b) over 2 months when stored at 37° C. A score of 1 is no change while 10 is highest change in color.

FIG. 5 shows the texture change in a topical ointment composition comprising fine grain or standard grain L. reuteri culture powder with desiccant (a) or without desiccant (b) over 2 months when stored at 37° C. Increasing coarseness score is equivalent to increasing feeling of the culture powder and grainy clumpy texture.

FIG. 6 shows a bar chart showing the mean reduction in SCORAD of the subjects after continuous use of the probiotic (ointment composition comprising fine grain L. reuteri culture powder) or control product (anhydrous base formulation) at baseline, Visit 2 (day 28) and Visit 3, end of study visit (day 56).

FIG. 7 shows a bar chart showing the mean reduction in Local SCORAD of the subjects after continuous use of the probiotic (ointment composition comprising fine grain L. reuteri culture powder) or control product (anhydrous base formulation) at baseline, Visit 2 (day 28) and Visit 3, end of study visit (day 56).

FIG. 8 shows a Graph A displaying the mean dryness score of Intensity of lesions score for dryness (SCORAD) of the subjects in each group for each time point. Graph B displays the mean dryness (local SCORAD) of the subjects for the probiotic product (ointment composition comprising fine grain L. reuteri culture powder) and the control product (anhydrous base formulation).

### DETAILED DESCRIPTION OF THE INVENTION

### Definitions

All words and terms used herein shall be considered to have the same meaning usually given to them by the person skilled in the art unless another meaning is apparent from the context.

The singular “a” and “an” shall be construed as including also the plural. Compositions “comprising” one or more recited elements may also include other elements not specifically recited.

Herein, the term “treatment” may encompass both the alleviation of symptoms as well as preventing the onset of symptoms. Accordingly, this term includes prevention, reduction and prophylaxis of a condition or a disorder, or the like. The term “disorder” may also include a disease.

Whenever the term “bacteria” is used herein, this is intended to include lactic acid producing bacterial strains, but it is not limited to any particular strain.

A “container” as referred to herein, is intended to mean any type of reservoir, box or carton, tube or the like for storing the ointment composition comprising the probiotic bacteria and the desiccant polymer present on a solid support, wherein said reservoir as far as possible provides a shield from surrounding moisture, light and air/oxygen. Said container may have any shape and can be made of a suitable material as disclosed elsewhere herein, but it is not limited thereto. The container may also be made from a material that can incorporate the desiccant polymer. If so, no separate solid support (e.g. in the form of a strip or a film) will be required for providing the desiccant polymer in close contact with the ointment composition.

An “air-tight” container as referred to herein is intended to mean a container that is air-tight as far as possible, i.e. that will prevent air/oxygen and/or moisture from entering the container which may negatively affect the properties of the ointment composition.

However, this does not mean that no air/oxygen/moisture at all will enter said container. Instead, it means that it will be made from a material that aim to prevent air/oxygen/moisture from entering the container and reaching the ointment composition.

An “anhydrous base formulation” as disclosed herein forms part of the ointment composition and comprises a vegetable fat, a vegetable oil and a hydrogenated vegetable oil. Examples of such vegetable fats, vegetable oils and hydrogenated vegetable oils suitable in the context of the present disclosure can be found elsewhere herein.

The anhydrous base formulation may be defined as a solid, or rather semi-solid, phase at room temperature (about 25° C.), said formulation being similar to a lotion or cream or the like at room temperature. It may also be referred to as an emollient, ointment, salve, balm or paste. Accordingly, in one aspect herein, the anhydrous base formulation may be a semi-solid phase formulation. It may also in another aspect herein be a solid phase formulation. In yet another aspect, the anhydrous base formulation may be in a phase between a solid phase and a semi-solid phase. Importantly, the anhydrous base formulation is not a liquid, such as a liquid oil. Instead, it is a non-liquid composition at room temperature (about 25° C.). The skilled person can easily distinguish between the different phases as described herein. Usually, a semi-solid phase composition means that the part constituting the vegetable oil constitutes less than or about 50% of the total anhydrous base formulation. This means that the part comprising the vegetable fat and the hydrogenated vegetable oil together usually constitutes more than or about 50% of the total anhydrous base formulation.

Hence, the relative contents of the vegetable fat, a vegetable oil and a hydrogenated vegetable oil of the anhydrous base formulation are such that the anhydrous base formulation obtains a texture as described herein. Examples of relative amounts of the ingredients are provided elsewhere herein. There are also provided herein other anhydrous base formulations in a broader sense.

No water is added to the anhydrous base formulation during its preparation. However, the base formulation still contains some small amounts of water as water forms part of the ingredients included in the formulation. In this regard, “anhydrous” is intended to mean that no water has been added to the formulation during its preparation and that the formulation per se comprises only small amounts of water or moisture, i.e. the formulation is essentially water-free.

An “ointment composition” as referred to herein, comprises an anhydrous base formulation and a fine grain culture powder of dried viable probiotic bacteria having a particle size distribution of d(90)<250 μm. The ointment composition referred to herein is also sometimes simply referred to as a topical probiotic product, a probiotic product, a topical ointment composition comprising probiotic bacteria, or the like.

The texture of the ointment composition is similar to a cream or a lotion or the like, or as described elsewhere herein. The texture of the anhydrous base formulation and the ointment composition can be described as very similar as the combination of the fine grain culture powder and the components of the anhydrous formulation surprisingly complemented each other to result in a smooth texture excellent for topical application. Hence, the ointment composition may also be defined herein as a solid or semi-solid formulation, in accordance with the anhydrous base formulation. However, whenever an anhydrous base formulation is referred to herein, this means a formulation without the presence of probiotic bacteria therein.

No water has been added to an ointment composition of the present disclosure. Any water present in the composition originates from the defined ingredients of the ointment composition/anhydrous base formulation, i.e. the oil or the fats present therein. It may also be small amounts of water in the composition in the form of moisture that has been absorbed from the surroundings.

“Probiotic bacteria” or “probiotic” refers to live microorganisms, such as bacteria that provide health or well-being benefits to a user, such as when they are consumed or used in any other way. The effect can e.g. be mediated by improving or restoring the flora of the gut when such products are consumed. There are also topical products comprising probiotic bacteria providing beneficial effects to the skin of a user, although the majority of topical products containing bacteria have used bacterial lysates instead of live bacteria.

Accordingly, the probiotic bacteria may form part of different types of products, such as food products, medicines or cosmetics.

A “fine grain culture powder of viable probiotic bacteria” as referred to herein, is a powder of dried bacteria that has been presented with a certain particle size distribution providing the powder with a certain texture.

A fine grain culture powder of probiotic bacteria used herein has a particle size distribution of d(90)<250 μm. This “mean” particle size is smaller than a standard culture powder residing in the range of d(90)<600 μm. It is sometimes also referred to as “a fine grain culture powder”, “a fine grain culture powder of probiotic bacteria”, or the like.

A dried fine grain culture powder as defined herein may be provided in the form of a freeze-dried, spray-dried or vacuum-dried powder. In particular, it may be provided in the form of a “freeze-dried” powder.

A “vegetable fat” is solid at room temperature and comprises fats extracted from seeds but also from other parts of fruits. Vegetable fats are mixtures of triglycerides. Vegetable fats are usually edible. Usually, vegetable fats are defined as having a fat content of more than 90%.

A “vegetable oil” is a liquid at room temperature and comprises oils extracted from seeds but also from other parts of fruits. Vegetable oils are mixtures of triglycerides. Vegetable oils are usually edible. Usually, vegetable oils are defined as having a fat content of more than 90%.

A “hydrogenated vegetable oil” is a vegetable oil that has been chemically altered to become solid at room temperature. Hydrogenated oil is more stable against rancidity during storage than vegetable oil. Usually, hydrogenated vegetable oils are defined as having a fat content of more than 90%.

A “desiccant polymer” as referred to herein is a polymer providing moisture absorbing properties when present on or incorporated into a solid support rendering it possible for such a material to absorb any moisture present in an ointment composition according to the present disclosure. Examples of desiccant polymers are provided elsewhere herein, but the present disclosure is not limited thereto.

A “solid support” as referred to herein, is a support of a suitable solid material as exemplified elsewhere herein to which a desiccant polymer has been absorbed or incorporated. The solid support may also form part of the container per se.

“Topical” administration or for “topical use” when referred to in combination with an ointment composition of the present disclosure means that the ointment composition is applied onto a (tissue) surface, most commonly a skin surface, of a human or an animal.

“Dried bacteria” can be achieved in different ways, including but not limited to freeze-drying, spray-drying, spray-freeze drying and vacuum drying bacteria. In one particular embodiment drying refers to freeze-drying.

The tissue surface absorbs at least a part of the ointment composition which can thereby provide beneficial properties to the tissue, such as softness of the skin and/or possibly effects in the form of reducing dryness and itchiness as described elsewhere herein.

### DETAILED DESCRIPTION

For topical products (including topical cosmetic products) the appearance, such as smell and color, and texture is of great importance, but this is a problem when working with live probiotic bacteria. There are many limitations of using probiotics in e.g. cosmetic formulations, due to the instability of live bacteria as an ingredient in product for storage due to their sensitivity to moisture. Some products have overcome this obstacle by instead using dead bacteria (such as bacterial lysates), bacterial substances or ferments where bacterial stability is not an issue.

However, if it is desired to use live bacteria, the instability of such foirt ulations requires that new formulations and packaging solutions need to be invented and produced. When using dried (e.g. freeze dried) culture powders in topical formulations, they may become grainy and lumpy, and the user would then feel the culture powder when rubbing the formulation into the skin. This consistency alteration would cause an unpleasant sensation when rubbing the formulation onto the skin, especially for subjects with sensitive skin.

The present disclosure presents a way of improving the physical characteristics of the topical ointment composition in addition to providing suitable ways for its storage.

Herein it has been identified a way to reduce the lumpiness or graininess of an ointment composition comprising dried viable probiotic bacteria, by grinding the dried bacterial powder into particles with a smaller diameter than standard grain particles. It was shown herein that using a finer grain culture powder of probiotic bacteria as compared to a standard powder of probiotic bacteria in the ointment compositions generated an ointment composition with a smooth and improved texture resulting in a more comfortable experience when topically applying the composition onto the skin of the user. Hence, using a fine grain powder as described herein resulted in less lumps forming and reduced graininess, as compared to a standard grain powder.

Using a fine grain powder solved the problem with the sensation of the composition when rubbing it onto the skin, but this solution in turn caused new issues, where the exposure of the moisture sensitive bacteria to its surrounding environment, and to the presence of small but deleterious amounts of water, is increased. This may in turn result in decreased viability of the probiotic bacteria due to the increased contact with the small amounts of water or moisture that is present in the composition.

When moisture sensitive dried (e.g. freeze-dried) bacteria come into contact with water, they will exit their dormant state which they are in when dried, and they will instead become activated. This causes additional problems with bacterial viability, since no or little nutrients are available within the formulation. It also causes problems with change in color (discoloration) and smell of the formulation (odor) since bacterial metabolites and/or other molecules produced and released/secreted by the active bacteria may negatively affect the formulation and cause an unpleasant smell, texture and discoloration.

However, the combination of the ointment composition comprising a fine grain culture powder of dried viable probiotic bacteria with the desiccant polymer present on a solid support was surprisingly proven successful to overcome or at least mitigate obstacles with topical probiotic products in the prior art, the desiccant polymer to a large extent being capable of absorbing moisture from the ointment composition comprising the fine grain culture powder of probiotic bacteria. Furthermore, this combination was shown to minimize color and smell changes in the ointment composition, while at the same time maintaining viability of the probiotic bacteria present therein.

The ointment composition is provided in a container together with a desiccant polymer present on a solid support. Surprisingly, the desiccant polymer was capable of reducing moisture in the ointment composition leaving the probiotic bacteria present therein viable for a much longer period than expected.

Due to the characteristics of the ointment composition, it was not at all expected that a desiccant polymer placed in contact with the ointment composition would be capable of absorbing moisture to such an extent. As an example, due to the high viscosity of the ointment composition, which is due to the high content of vegetable fat and hydrogenated vegetable oil of the anhydrous base formulation, it was not expected to work in synergy with the desiccant polymer. Desiccant polymers are generally used for the gas phase, e.g. in a bottle neck for passage with capsules, or for liquid solutions and would not be expected to work in a solid or semi-solid formulation based on the diffusion properties of such high viscosity formulas. This is mainly due to that the diffusion rate of water within a solid or semi-solid formulation is markedly lower compared to in a soluble, or liquid, formulation.

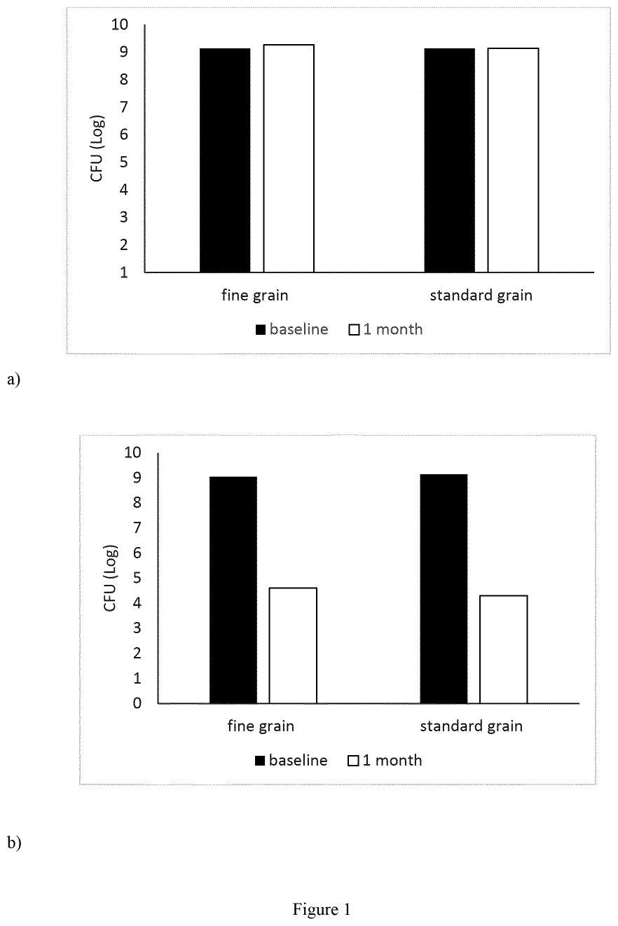

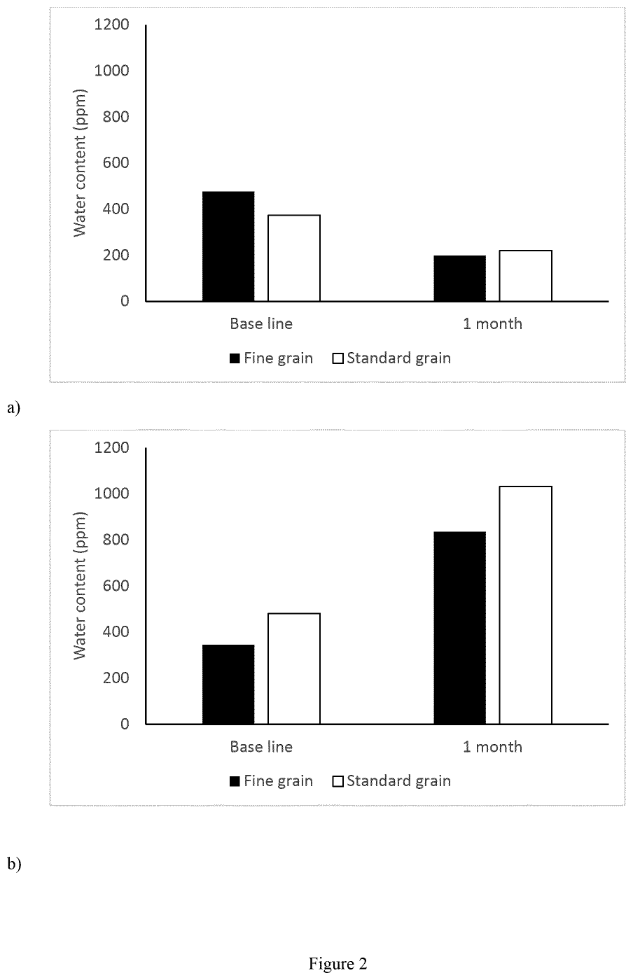

Accordingly, as shown herein, the survival of probiotic bacteria during storage, including but not limited to Lactobacillus reuteri DSM 17938, was shown to be markedly higher with a desiccant polymer present compared to when a desiccant is not present during storage (FIG. 1). Surprisingly, this was true also for the ointment composition comprising the fine grain culture powder of probiotic bacteria as shown in FIG. 1a. This was despite the fine grain culture powder likely being more prone to absorb moisture than a standard grain culture powder would be, as further explained herein. A lower water content is associated with a more stable product and as further shown in FIG. 2, the water content was decreased during 1 month of storage when a desiccant was present and the results appeared to be most obvious when a fine grain culture powder was used compared to standard culture powder. The surprising properties of the ointment composition stored together with the desiccant polymer was further confirmed in a long-term stability study illustrated in Example 4. The results show that the stored samples of the ointment composition possess good long-term stability capabilities as the viability of the bacteria is high and the appearance and the sensory results are satisfactory.

Accordingly, there is provided herein a container comprising: i) an ointment composition comprising an anhydrous base formulation and a fine grain culture powder of dried (e.g. freeze-dried) viable probiotic bacteria having a particle size distribution of d(90)<250 μm, wherein said anhydrous base formulation comprises a vegetable fat, a vegetable oil and a hydrogenated vegetable oil; and ii) a solid support comprising a desiccant polymer, wherein said anhydrous base formulation is a solid or semi-solid phase formulation at about 25° C.

A fine grain culture powder of dried viable probiotic bacteria may be provided in the form of e.g. a freeze-dried, spray-dried or vacuum-dried powder. In particular, it may be provided in the form of a “freeze-dried” powder.

The solid support comprising or incorporating a desiccant polymer serves the purpose of acting as a moisture absorbing material to reduce any moisture already present in the formulation and/or entering the container affecting the ointment composition and the viability of the probiotic bacteria present therein.

Surprisingly, despite the properties and texture of the ointment composition as previously discussed herein the desiccant was shown to be very efficient in reducing moisture in the composition. The packaging of the ointment composition is in a (tight) container that does not allow the passage of air/oxygen or water/moisture into the composition. The desiccant polymer is placed in direct contact with the ointment composition to be most efficient in moisture absorbing. The container may be any suitable container, possibly rigid, of a material that does not (in essence) allow oxygen or water to enter the container.

The container includes but is not limited to a container made of glass, aluminum, plastic, polymers, cork, rubber, or flexible multi-layer laminates, or a combination thereof or as described elsewhere herein. The container may also be made of polyethylene theraphthalate glycol. Said container may also be made of aluminum, glass, plastic, cork, rubber or a combination thereof.

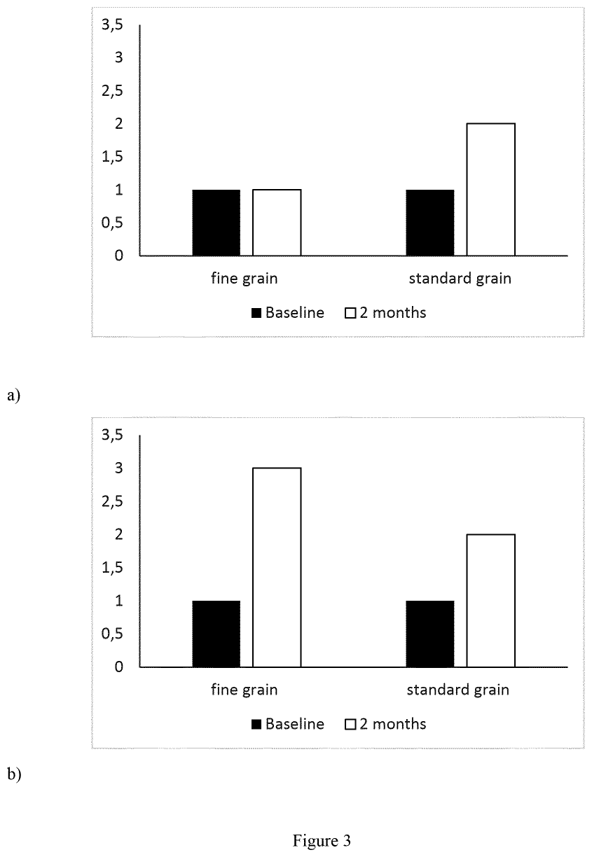

As shown in FIG. 3b, both fine grain and standard grain culture powder without desiccant show increased changes in odor during storage (which is not desired), compared to than when the desiccant is present (FIG. 3a). As shown in FIG. 3a, the effect on odor development was surprisingly even greater with the ointment composition comprising the fine grain culture powder, in essence preventing odor development during the first two months of storage.

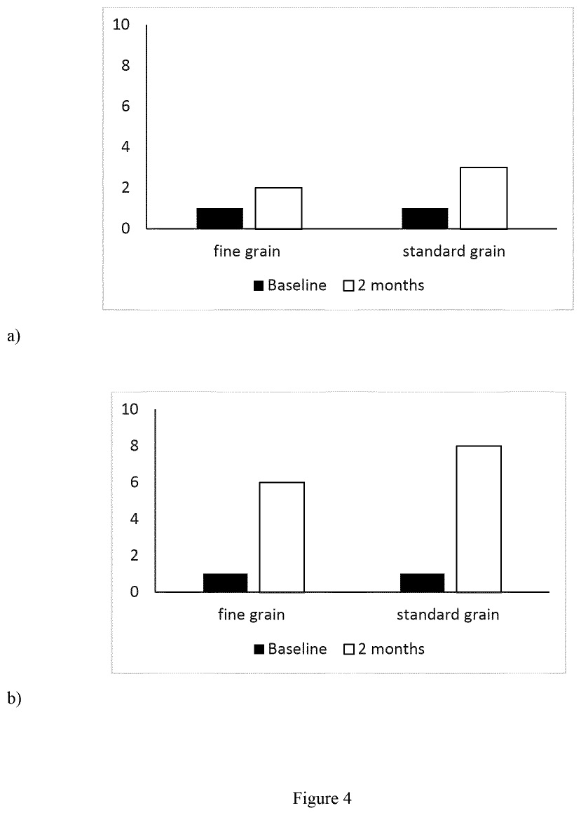

A similar effect was seen on color change (discoloration), although again fine grain culture powder was unexpectedly shown to be superior to standard grain culture powder both with and without desiccant polymer, but particularly with desiccant (FIG. 4).

Notably, when the ointment composition comprising the fine grain culture powder has been stored with the desiccant polymer for 1 month, a significant amount of the water appears to have been absorbed by the desiccant polymer also in comparison to the standard grain powder (FIG. 2a). Without the desiccant polymer, the amount of water in both compositions increase during storage, which will negatively affect the stability of the compositions (FIG. 2b).

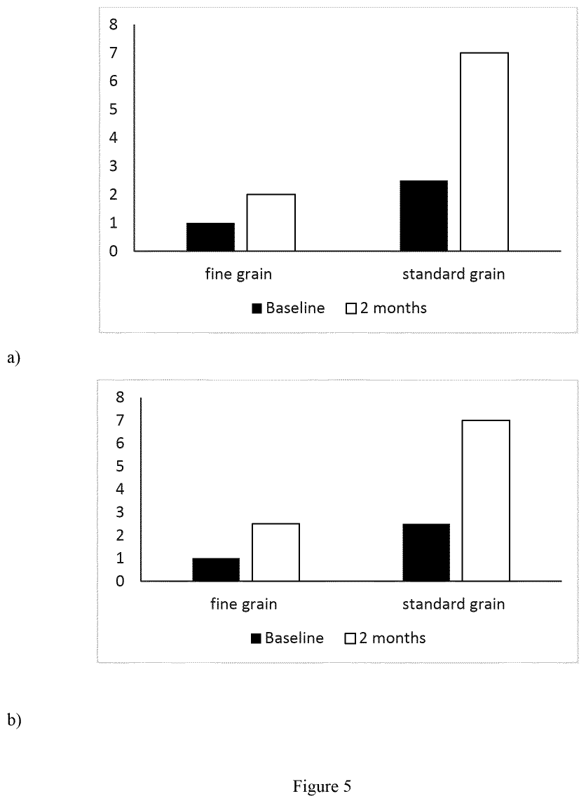

In terms of texture, the fine grain culture powder is also superior to standard culture powder since it is smoother at baseline and after 2 months, both with and without desiccant (FIG. 5). The standard culture powder becomes grainy and lumpy and one can therefore feel the culture powder when rubbing the ointment composition onto the skin, which does not make it suitable for topical use.

Accordingly, it was shown herein that it was not only possible to increase the stability, but at the same time also provide an ointment composition with less discoloration and change in odor and texture over time, which is of importance for topical formulations in general, and particularly for cosmetic products.

There is also provided herein a container that is substantially airtight. In such an airtight container, the ointment composition comprising the fine grain probiotic culture powder is in direct contact with the desiccant polymer present on a solid support and the components are packed tight together that will not (in essence) allow air/oxygen or water/moisture to enter the container. Of course, the container will not be completely airtight and some air/oxygen and water/moisture may enter. However, the container is manufactured from a material suitable for this purpose.

The anhydrous base formulation of an ointment composition present herein comprises a vegetable fat, a vegetable oil and a hydrogenated vegetable oil. The anhydrous base formulation is a solid or semi-solid phase formulation at about 25° C. as further described elsewhere herein. Accordingly, the anhydrous base formulation can also be described as a semi-solid phase as it has the texture of a cream or a lotion or the like at this temperature. It can also be described as a solid phase formulation if the contents of solids, i.e. vegetable fats and the hydrogenated vegetable oil is particularly high in the composition. Furthermore, in this context it means that no water has been added to the formulation during its preparation.

Accordingly, any water that is present in the formulation originates from the constituents of the formulation, i.e. the vegetable fat, the vegetable oil and/or the hydrogenated vegetable oil. The water content in an anhydrous base formulation is expected to be in the order of <500 ppm with desiccant or <1000 ppm i.e. 0.1% without the desiccant, but this may also vary.

A vegetable fat as disclosed herein may be selected from the group consisting of shea butter, aloe butter, avocado butter, apricot butter, cocoa butter, coffee bean butter, cupuacu butter, refined butter, hemp seed butter, illipe butter, kokum butter, macadamia nut butter, mango butter, mochacchino butter, murumuru butter, olive butter, peach butter, pistachio nut butter, coconut butter, shealoe butter, canola butter, soybean butter, sunflower butter, corn butter, palm butter, palm-kernel butter, cotton seed butter, peanut butter and almond butter, particularly shea butter, but is not limited thereto. Particularly, a vegetable fat may be selected from shea butter, coconut butter, hemp seed butter, almond butter and olive butter.

A vegetable oil as disclosed herein may be selected from the group consisting of canola oil, soybean oil, sunflower oil, corn oil, peanut oil, cottonseed oil, olive oil, avocado oil, grapeseed oil, macadamia-nut oil, coconut oil, argan oil, wheat-germ oil, jojoba oil, palm-kernel oil, palm oil, chia oil, sesame oil, hemp oil, maringa oil, marula oil, acai oil, evening-primrose oil, calendula oil, ground-nut oil, brazilnut oil, safflower oil, olus oil, MCT oil, rose-hip seed oil, almond oil, hazelnut oil, rice-bran oil, pumpkin seed oil, baobob oil, cotton-seed oil, mustard seed oil, apricot kernel oil, castor oil, paradise nut oil, camelina oil, black cumin seed oil, broccoli seed oil, and carrot oil, particularly canola oil, but is not limited thereto. Particularly, a vegetable oil may be selected from canola oil, sunflower oil, coconut oil, palm oil, olive oil and argan oil.

A hydrogenated vegetable oil as disclosed herein may selected from the group consisting of canola oil, soybean oil, sunflower oil, corn oil, peanut oil, cotton seed oil, olive oil, avocado oil, grapeseed oil, macadamia-nut oil, coconut oil, argan oil, wheat-germ oil, jojoba oil, palm-kernel oil, palm oil, chia oil, sesame oil, hemp oil, maringa oil, marula oil, acai oil, evening-primrose oil, calendula oil, ground-nut oil, brazilnut oil, safflower oil, olus oil, MCT oil, rose-hip seed oil, almond oil, hazelnut oil, rice-bran oil, pumpkin seed oil, baobob oil, cotton-seed oil, mustard seed oil, apricot kernel oil, castor oil, paradise nut oil, camelina oil, black cumin seed oil, broccoli seed oil, and carrot oil, particularly canola oil or rapeseed oil, but is not limited thereto. Particularly, a hydrogenated vegetable oil may be selected from hydrogenated canola oil, sunflower oil, soybean oil, palm-kernel oil, avocado oil, olive oil, almond oil, jojoba oil and argan oil.

A particular hydrogenated vegetable oil for use in an ointment composition of the present disclosure is available from Dr. Straetmans GmbH (dermofeel® viscolid or dermofeel® viscolid palm oil free, Dr. Straetmans GmbH Merkurring 90 D-22143 Hamburg, Germany). The raw material for the hydrogenated vegetable oil varies and can e.g. be rapeseed or sunflower oil.

An anhydrous base formulation provided herein may comprise 20% to 80%, such as 30% to 70%, 30% to 60%, 40% to 80%, 45% to 80%, 50% to 80%, 60% to 80%, 70% to 80%, 40% to 60%, or 45% to 55%, by weight of a vegetable fat; 20% to 80%, such as 20% to 50%, 20% to 45%, 20% to 40%, 20% to 30%, 30% to 70%, 30% to 60%, 40% to 50%, 40% to 60%, or 40% to 50%, by weight of a vegetable oil; and 0.1% to 15%, such as 0.1% to 10%, 0.1% to 5%, 0.5% to 10%, or 0.5% to 5%, by weight of a hydrogenated vegetable oil. Naturally, the total amount of the components of said anhydrous base formulation amounts to 100% (total anhydrous base formulation). In some aspects, the vegetable fat and the hydrogenated vegetable oil are to a certain part exchangeable, e.g. meaning that a part of the vegetable fat of the anhydrous base formulation may be replaced with or exchanged with hydrogenated vegetable oil, or vice versa. Herein, the amounts referred to above may specifically refer to an anhydrous formulation wherein said vegetable fat is shea butter, said vegetable oil is canola oil and said hydrogenated vegetable oil is a hydrogenated rapeseed or a hydrogenated canola oil. Said anhydrous base formulation is provided in an ointment composition together with a fine grain culture powder of probiotic bacteria as described elsewhere herein.

An anhydrous base formulation presented herein may also comprise 45% to 55%, such as about 46%, 47%, 48%, 49%, 50%, 50.5%, 51%, 51.5%, 52%, 53%, or 54%, particularly about 50.5% by weight of a vegetable fat, such as shea butter; 40% to 50%, such as about 41%, 42%, 43%, 44%, 45%, 46%, 47.0%, 48%, 49%, particularly about 47% by weight of a vegetable oil, such as canola oil; and 0.5% to 10%, such as about 0.5% to 5%, such as 1%, 2%, 2.5%, 3%, 3.5%, 4%, 5%, 6%, 7%, 8%, or 9%, particularly about 2.5% by weight of hydrogenated vegetable oil, such as hydrogenated rapeseed or canola oil.

A solid support as disclosed herein, on which a (chemical) desiccant polymer is present or incorporated may be selected from the group consisting of a strip, a pad, a bead or a film, or the like, such as a foil. It may also be present as a moulding in a packaging material of said container. The latter means that no separate solid support is provided with the desiccant polymer, but the desiccant polymer is present within, or incorporated into the container material and is hence introduced during manufacture of the container.

The solid support comprising the desiccant polymer is positioned in the container in such way that it will be in direct contact with the ointment composition containing the probiotic bacteria so that it can thereby absorb any moisture from the composition. As mentioned above, the solid support comprising the desiccant polymer can also be provided by internally coating, or incorporating, the container used for the storage of the probiotic bacteria powder formulations with the desiccant polymer.

Herein, a specific desiccant polymer film (M-0026 Activ-Film, CSP Technologies, Auburn, USA) or a film with a desiccant material incorporated (as described in U.S. Pat. No. 8,003,179, for example PET12PE/ALU 12/PE/PE+desiccant/PE from Alcan) may be used, but the present disclosure is not limited thereto. Naturally, other desiccant polymers with a similar function may be used in the present disclosure.

When a solid support in the form of a strip comprising a desiccant polymer is used and placed in a container (e.g. in the form of a bottle or a tube) said strip may be about 0.1-10 mm thick, 1-30 mm wide and 1-80 mm long, but is not limited thereto. E.g. for a 25 ml tube the target is to have a minimum moisture capacity of the desiccant polymer strip of 60 mg at 22° C. and 80% RH (Relative Humidity). A minimum moisture capacity for other tube or container sizes may e.g. be determined based on this information and based on knowledge of the skilled persons of the water content of the raw material of the probiotic product. Any moisture absorbing capacity of a desiccant polymer will have a positive effect on the final probiotic product.

The final content of dried (e.g. freeze-dried) probiotic bacteria present in said ointment composition may be about 0.001 to 10%, such as 0.01 to 10%, 0.01% to 8%, or 0.01% to 5% by weight of the total ointment composition, particularly about 1% by weight of the total ointment composition. The ointment composition comprises the anhydrous base formulation and the fine grain probiotic bacteria culture powder.

Probiotic bacteria as referred to herein may be moisture sensitive bacteria as further defined herein. Probiotic bacteria may be probiotic lactic acid producing bacteria (such as lactobacilli and bifidobacteria), sometimes also referred to as lactic acid bacteria or lactobacillales. Lactic acid producing bacteria are a clade of Gram-positive, low-GC, acid-tolerant, generally nonsporulating, nonrespiring, either rod- or cocci-shaped bacteria that share common metabolic and physiological characteristics. These bacteria produce lactic acid as the major metabolic end product of carbohydrate fermentation. Genera that comprise the lactic acid producing bacteria include Lactobacillus, Leuconostoc, Pediococcus, Lactococcus, Bifidobacterium and Streptococcus. Currently preferred genus are Lactobacillus and Bifidobacterium. The genus may be Lactobacillus. Lactobacillus include several species including L. acetotolerans, L. acidifarinae, L. acidipiscis, L. acidophilus, L. agilis, L. algidus, L. alimentarius, L. amylolyticus, L. amylophilus, L. amylotrophicus, L. amylovorus, L. animalis, L. antri, L. apodemi, L. aviaries, L. bifermentans, L. brevis, L. buchneri, L. camelliae, L. casei, L. catenaformis, L. ceti, L. coleohominis, L. collinoides, L. composti, L. concavus, L. coryniformis, L. crispatus, L. crustorum, L. curvatus, L. delbrueckii subsp. bulgaricus, L. delbrueckii subsp. elbrueckii, L. delbrueckii subsp. lactis, L. dextrinicus, L. diolivorans, L. equi, L. equigenerosi, L. farraginis, L. farciminis, L. fermentum, L. fornicalis, L. fructivorans, L. frumenti, L. fuchuensis, L. gallinarum, L. gasseri, L. gastricus, L. ghanensis, L. graminis, L. hammesii, L. hamster, L. harbinensis, L. hayakitensis, L. helveticus, L. hilgardii, L. homohiochii, L. iners, L. ingluviei, L. intestinalis, L. jensenii, L. johnsonii, L. kalixensis, L. kefiranofaciens, L. kefiri, L. kimchi, L. kitasatonis, L. kunkeei, L. leichmannii, L. lindneri, L. malefermentans, L. mali, L. manihotivorans, L. mindensis, L. mucosae, L. murinus, L. nagelii, L. namurensis, L. nantensis, L. oligofermentans, L. oris, L. panis, L. pantheris, L. parabrevis, L. parabuchneri, L. paracasei, L. paracollinoides, L. parafarraginis, L. parakefiri, L. paralimentarius, L. paraplantarum, L. pentosus, L. perolens, L. plantarum, L. pontis, L. protectus, L. psittaci, L. rennini, L. reuteri, L. rhamnosus, L. rimae, L. rogosae, L. rossiae, L. ruminis, L. saerimneri, L. sakei, L. salivarius, L. sanfranciscensis, L. satsumensis, L. secaliphilus, L. sharpeae, L. siliginis, L. spicheri, L. suebicus, L. thailandensis, L. ultunensis, L. vaccinostercus, L. vaginalis, L. versmoldensis, L. vini, L. vitulinus, L. zeae, and L. zymae.

Currently preferred examples of such probiotic bacterial strain include Lactobacillus reuteri strains, such as Lactobacillus reuteri DSM 17938, Lactobacillus reuteri ATCC PTA 6475, Lactobacillus reuteri ATCC PTA 5289, Lactobacillus reuteri DSM 32846, Lactobacillus reuteri DSM 32847, Lactobacillus reuteri DSM 32848, Lactobacillus reuteri DSM 32849, Lactobacillus reuteri DSM 27131 or Lactobacillus reuteri DSM 32465.

Lactobacillus reuteri DSM 17938 was deposited under the Budapest Treaty at the DSMZ-Deutsche Sammlung von Mikroorganismen und Zellkulturen GmbH (Mascheroder Weg 1b, D-38124 Braunschweig, Germany) on Jan. 30, 2006.

Lactobacillus reuteri ATCC PTA 6475 was deposited under the Budapest Treaty at the American Type Culture Collection (10801 University Blvd., Manassas, Va. 20110-2209, U.S.) on Dec. 21, 2004.

Lactobacillus reuteri DSM 27131 was deposited under the Budapest Treaty at the Leibniz Institut DSMZ-Deutsche Sammlung von Mikroorganismen und Zellkulturen GmbH (Inhoffenstr. 7B, D-38124 Braunschweig, Germany) on Apr. 18, 2013.

Lactobacillus reuteri DSM 32846, DSM 32847, DSM 32848 and DSM 32849 were deposited under the Budapest Treaty at the Leibniz Institut DSMZ-Deutsche Sammlung von Mikroorganismen und Zellkulturen GmbH (Inhoffenstr. 7B, D-38124 Braunschweig, Germany) on Jul. 4, 2018.

Lactobacillus reuteri DSM 32465 was deposited under the Budapest Treaty at the Leibniz Institute DSMZ—Deutsche Sammlung von Mikroorganismen und Zellkulturen GmbH (Inhoffenstr. 7B, D-38124 Braunschweig, Germany) on Mar. 21, 2017.

Lactobacillus reuteri ATCC PTA 5289 was deposited under the Budapest Treaty at the American Type Culture Collection (10801 University Blvd., Manassas, Va. 20110-2209, U.S.) on Jun. 25, 2003.

Other examples of such probiotic bacterial strain include Lactobacillus gasseri, such as Lactobacillus gasseri DSM 27123 and Lactobacillus gasseri DSM 27126. These strains have been deposited at DSMZ (Leibniz Institute DSMZ—Deutsche Sammlung von Mikroorganismen und Zellkulturen GmbH, Inhoffenstr. 7B, D-38124 Braunschweig, Germany) on Apr. 18, 2013.

Accordingly, probiotic bacteria for use herein may be lactic acid producing bacteria, optionally lactic acid producing bacteria of the genus Lactobacillus, such as a Lactobacillus reuteri strain (now also known as Limosilactobaillus reuteri), such as Lactobacillus reuteri DSM 17938 or Lactobacillus reuteri ATCC PTA 6475, or Lactobacillus gasseri, such as Lactobacillus gasseri DSM 27123 and Lactobacillus gasseri DSM 27126. Bifidobacterium include several species including B. longum and B. breve. The Bifidobacterium species may be B. longum.

Other examples of probiotic bacteria for use herein are Bifidobacterium longum DSM 32947, and Bifidobacterium longum (DSM 32948) (deposited under the Budapest Treaty at the Leibniz Institute DSMZ-Deutsche Sammlung von Mikroorganismen and Zellkulturen GmbH (Inhoffenstr. 7B, D-38124 Braunschweig, Germany) on Nov. 1, 2018).

It is also envisaged herein that combinations of different strains of bacteria may be used in a composition or formulation as described herein.

The desiccant polymer mentioned herein may be, but is not limited to, a desiccant polymer of a hygroscopic substance such as silica, activated charcoal, calcium sulfate, calcium chloride and molecular sieve or any combination thereof.

Making the fine grain culture powder can be performed with several size reduction techniques including but not limited to hammer mills, conical mills, ball mills and sieving. Any suitable method may be used to produce the fine grain culture powder disclosed herein.

The particle size distribution of the fine grain culture powder is in the range of d(90)<250 μm, such as d(90)<200 μm, or d(90)<150 μm measured using laser diffraction. The particle size distribution of a standard culture powder is generally d(90)<600 μm. “d(90)” in this context, means that 90% of the particles have a diameter below the number following the d(90). This is a common way to describe particle size distribution using Laser Diffraction (LD) and is well-known to a person skilled in the art.

As mentioned elsewhere herein, the present disclosure is also directed towards the cosmetic use of an effective amount of at least one probiotic bacteria disclosed herein, especially of the Lactobacillus and/or Bifidobacterium sp. genus and, in particular, of the Lactobacillus reuteri strain, such as the Lactobacillus reuteri DSM 17938 strain.

An ointment composition according to the present disclosure may generally comprise from 10² to 10¹² cfu/g, in particular from 10⁵ to 10¹⁰ cfu/g, and more particularly from 10⁷ to 10¹⁰ cfu/g, such as 10⁶, 10⁸, 10⁹ or 10¹¹, cfu/g of probiotic bacteria, such as lactic acid producing bacteria.

There is also provided herein an ointment composition comprising an anhydrous base formulation and a fine grain culture powder of dried viable probiotic bacteria with a particle size distribution of d(90)<250 μm, wherein said anhydrous base formulation comprises a vegetable fat, a vegetable oil and a hydrogenated vegetable oil and wherein said anhydrous base formulation is a solid or a semi-solid phase formulation at about 25° C.

As previously mentioned herein, said anhydrous base formulation of the ointment composition may comprise in some aspects: i) 20% to 80%, such as 45% to 80% or 45% to 55%, by weight of a vegetable fat; ii) 20% to 80%, such as 20% to 50% or 40% to 50%, by weight of a vegetable oil; and iii) 0.1% to 15%, such as 0.5% to 10%, by weight of a hydrogenated vegetable oil.

In some aspects, the anhydrous base formulation of the ointment composition may comprise: i) 45% to 80% by weight of a vegetable fat; ii) 20% to 50% by weight of a vegetable oil; and iii) 0.1% to 15% by weight of a hydrogenated vegetable oil. Further in this regard, said vegetable fat may be shea butter, said vegetable oil may be canola oil and said hydrogenated vegetable oil may be hydrogenated canola oil or hydrogenated rapeseed oil.

Yet further in this regard, said anhydrous base formulation may comprise: i) 45% to 55%, such as about 50.5% by weight of shea butter; ii) 40% to 50%, such as about 47% by weight of canola oil; and iii) 0.5% to 10%, such as about 2.5% by weight of hydrogenated rapeseed oil or hydrogenated canola oil. As mentioned elsewhere herein, there is also provided an anhydrous base formulation comprising about 50.5% by weight of a vegetable fat, such as shea butter; about 47.0% by weight of a vegetable oil, such as canola oil; and about 2.5% by weight of a hydrogenated vegetable oil, such as hydrogenated canola oil.

Other ranges falling within these ranges are also applicable as mentioned and disclosed elsewhere herein in relation to an anhydrous base formulation discussed in the context of the present disclosure.

The final content of dried (e.g. freeze-dried) probiotic bacteria in said composition may be about 0.001% to 10%, such as 0.01% to 5% by weight, such as about 1% per weight of the total ointment composition, or as described elsewhere herein. As also described elsewhere herein, said probiotic bacteria may be lactic acid producing bacteria in said ointment composition, optionally Lactobacillus, such as a Lactobacillus reuteri strain, such as Lactobacillus reuteri DSM 17938. Other examples of suitable probiotic bacteria for an ointment composition are also described herein. Said probiotic bacteria may be moisture sensitive bacteria.

There is also provided herein the use of an ointment composition for non-therapeutic topical administration to a skin surface of a subject, such as a human or an animal. Such administration may have a cosmetic effect which is not therapeutic but beneficial to the well-being of the subject in question.

As shown in example 3, the ointment composition of the present disclosure fulfilled cosmetic acceptability when used in adults also resulting in a soft sensation when applied to the skin. Accordingly, the ointment composition of the present disclosure is suitable for cosmetic use as a lotion or cream or the like to be applied to the skin of a user.

There is also provided an ointment composition as disclosed herein for use in therapeutic topical administration to a skin surface of a subject, such as a human or an animal. Accordingly, there is provided herein an ointment composition for use as a medicament.

In this regard, there is provided an ointment composition for use in a method of treating or preventing atopic dermatitis. There is also provided an ointment composition as disclosed herein for use in treating or preventing skin conditions or disorders. There is also provided herein an ointment composition for use in a method for treating or preventing vaginal conditions or disorders.

In addition, an ointment composition as provided herein may be for use in a method of treating or preventing, including but not limited to atopic or eczema prone-skin, dry skin, normal, psoriasis, rosacea, sensitive, reactive skin, oily skin, nappy rash, dermatitis, seborrheic dermatitis, dandruff, scarring, stretch marks, impetigo, and allergy. There is also provided the use of an ointment composition in the manufacture of a medicament for the treatment or prevention of skin conditions or disorders, such as atopic dermatitis. In addition, there is provided a method for treating or preventing skin conditions or disorders, such as atopic dermatitis, said method comprising topically, such as to a skin or a mucosal surface, administering a pharmaceutically or therapeutically effective amount of an ointment composition as disclosed herein to a subject in need thereof. There is also provided herein a method for therapeutic topical administration of an ointment composition as disclosed herein, said method comprising topically applying or administering a pharmaceutically or therapeutically effective amount of an ointment composition as disclosed herein to a subject in need thereof.

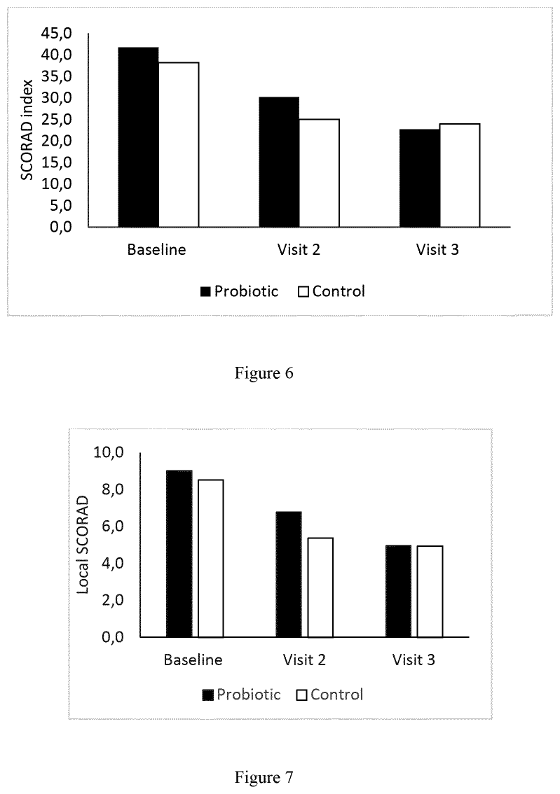

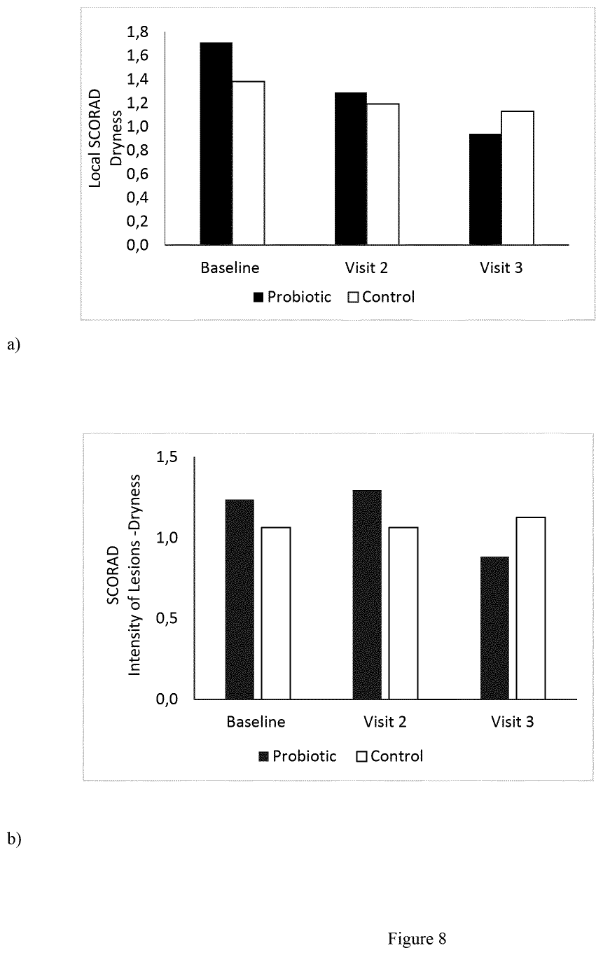

SCORing Atopic Dermatitis (SCORAD) using the standardized form from the European task force on Atopic Dermatitis was used when evaluating the effect of the ointment composition comprising the fine grain probiotic culture powder on patient suffering from this condition. As shown in example 3, a somewhat greater reduction in SCORAD and local-SCORAD was seen when using the probiotic product as compared to the control product (FIGS. 6 and 7). However, within the SCORAD- and local-SCORAD Intensity of Lesions score (Sum of Erythema, Edema, Oozing, Excoriation, Lichenification and Dryness), the difference was even more significant and a stronger reduction in dryness was shown when the probiotic product was used as compared to when the control product was used (FIG. 8).

The ointment composition for a therapeutic or non-therapeutic use may be provided together with a solid support comprising a desiccant polymer, optionally wherein said solid support is selected from the group consisting of a strip, a pad, a bead or a film, or is present as a moulding in a packaging material of a container for holding said composition.

A pharmaceutically or therapeutically effective amount of an ointment composition as disclosed herein means an amount that is efficient for a therapeutic effect to occur in a subject receiving a treatment with an ointment composition described herein. Such an effective amount will be determined by the skilled practitioner treating the subject in question. Provided herein are preferred concentrations of the probiotic bacteria present in the ointment composition also providing a guidance in this regard. It is also referred to herein an effective amount of an ointment composition of the present disclosure. Such an amount of an ointment composition means an amount that is needed to achieve any effect described, such as a cosmetic effect.

The ointment composition of the present disclosure may also be used to protect the normal bacteria, fungi and viruses that are present on the skin (the microbiome), to rebalance the microbiome, to have no effect on the normal inhabitants of the microbiome, to exclude opportunistic pathogens from inhabiting the skin and to allow the ointment composition provided herein to exert its effects on the skin which includes and is not limited to preventing skin infection and improving skin conditions.

The ointment composition of the present disclosure may also be used for hydrating the skin, protecting the skin, providing moisture, softening, decreasing redness, swelling, decreasing wrinkles and lines, smoothing pores, i.e. wherein said ointment composition has a cosmetic, non-therapeutic effect. Accordingly, there is provided herein an ointment composition for use in a method of treating or preventing any of the herein mentioned disorders, diseases or conditions.

There is also provided herein the use of an ointment composition as a cosmetic, optionally wherein said composition is provided together with a solid support comprising a desiccant polymer, optionally wherein said solid support is selected from the group consisting of a strip, a pad, a bead or a film, or is present as a moulding in a packaging material of a container holding said composition. This is also described elsewhere herein.

There is furthermore provided a method for preparing an ointment composition as disclosed herein, said method comprising the steps of: i) mixing an anhydrous base formulation comprising a vegetable oil, a vegetable fat and a hydrogenated vegetable oil as disclosed herein to at about 65 to 70° C. until the fats are melted; ii) cooling the mixture of step i) to about 30° ; and iii) mixing the anhydrous base formulation with a culture powder of dried viable probiotic bacteria to obtain an ointment composition. Variants of this method include mixing the anhydrous base formulation comprising a vegetable oil, a vegetable fat and the hydrogenated vegetable oil with the dried culture powder without using a heating step. There is also provided an ointment composition obtainable by such a method.

There is furthermore provided a method for prolonging the shelf life and/or improving the stability and/or for reducing smell/odor and/or color changes of an ointment composition disclosed herein comprising performing a method of preparing an ointment composition as described herein, and thereafter:
iv) obtaining the ointment composition of step iii) of the method of preparing an ointment composition and thereafter placing said composition in an air-tight container;
v) placing a solid support comprising a desiccant polymer in said container together with said ointment composition, optionally wherein said solid support is a strip, a pad, a bead or a film and/or wherein said solid support comprising a desiccant polymer is present in the packaging material of said container; and thereafter vi) sealing said container.

In this regard, there is also provided an air-tight container comprising an ointment composition and a desiccant obtainable by a method presented herein.

There is also provided the use of a solid support comprising a desiccant polymer as disclosed herein for improving the stability and/or shelf life of an ointment composition as disclosed herein. In addition, there is provided the use of a solid support comprising a desiccant polymer as disclosed herein for preventing changes in the color and/or smell of an ointment composition as disclosed herein. As explained elsewhere herein, the desiccant surprisingly increased the stability and/or shelf-life of the ointment composition when stored in contact with the composition. This assisted in maintaining the beneficial properties of the ointment composition, such as its color and it also prevented the development of unpleasant smell from the composition. Without wishing to be bound by theory, it is envisaged that these beneficial properties are related and may also be related to the stability of the ointment composition.

The present disclosure will now be illustrated and exemplified by the experimental section, but it is not intended to be limited thereto.

### EXPERIMENTAL SECTION

### Example 1—Manufacture of the Ointment Composition

### Anhydrous Base Formulation

The anhydrous base formulation was made by mixing shea butter (Lipex Sheasoft from AAK, Sweden) 50%, canola oil (Lipex Bassol C from AAK, Sweden) 46.5% and hydrogenated vegetable oil (Dermofeel viscolid from Dr Straetmans, Germany) 2.5% (the percentages are amounts of the final ointment composition, i.e. the ointment composition mentioned below that also includes 1% probiotic bacteria of the total composition) at 65-70° C. until the fats had melted and a homogenous solution was obtained. The mixture was then cooled down to 30° C.

The anhydrous base formulation used in this example may be considered a semi-solid formulation.

### Bacterial Culture Powder Preparation

Two different Lactobacillus reuteri culture powders were prepared; a fine grain Lactobacillus reuteri DSM 17938 freeze-dried culture powder with a particle size distribution of d(90)<150 μm and a standard grain Lactobacillus reuteri DSM 17938 freeze-dried culture powder with a particle size distribution of d(90)<600 μm. The culture powders were left to adjust to room temp for 30 minutes.

### Product Formulation

The anhydrous base formulation was weighed in a clean mixing jar and the freeze-dried bacteria powders were added to a final concentration of 1% (approximately 2×10^9 CFU/g) to obtain two ointment compositions, one with a fine grain culture powder of probiotic bacteria and one with a standard grain culture powder of probiotic bacteria. The samples were mixed thoroughly using a clean glass stirrer. In the final ointment composition, the amount of shea butter is 50%, the amount of canola oil is 46.5%, the amount of hydrogenated oil is 2.5% and the amount of L. reuteri DSM 17938 culture is 1%.

### Product Packaging and Desiccant Strip

The two different ointment compositions, based on the anhydrous base formulation mixed with either fine grain or standard grain Lactobacillus reuteri culture powder were transferred to clear 30 ml glass jars (Opella, Sweden, Article #: 50480400). A desiccant strip (45 mm×7.5 mm×1.6 mm, M-0026 Activ-film from CSP Technologies) was added to one jar for each culture type and all jars were then covered with a fitted hard plastic bakelite lid (Opella, Sweden, Article #: 51470400).

### Sample Components

The control sample was anhydrous base formulation on its own, with no culture added and without desiccant polymer. The test samples were prepared according to the following:
1. Lactobacillus reuteri DSM 17938 standard culture with desiccant.
2. Lactobacillus reuteri DSM 17938 standard culture powder without desiccant.
3. Lactobacillus reuteri DSM 17938 fine grain culture powder with desiccant.
4. Lactobacillus reuteri DSM 17938 fine grain culture powder without desiccant.

### Example 2—Stability and Sensory Study with Freeze Dried L. Reuteri DSM 17938, Fine Grain and Standard Grain Culture Powder

### Stability Testing

To assess the stability of each composition, samples of example 1 were collected at baseline and at 1-month intervals. At these time points, lg of each sample was removed from the sample vial and mixed thoroughly in a standardized medium, for example deMan Rogosa and Sharpe culture medium and a dilution series was made (in Phosphate buffered saline) cultured onto MRS agar plates. Plates were incubated at 37° C. anaerobically for 48 hours. The number of colony forming units (CFUs) was quantified from each sample, to assess the viability of the bacteria in each sample as a direct measurement of stability of each of the ointment compositions.

### Sensory Analysis

Sensory analysis (smell, color/appearance, texture) was also performed on each of the samples at baseline and at 1 to 2-month intervals. Unpleasant smell and color were graded on a scale of 1-10 where 1 was the base formulation at baseline measurement (1—no smell to 10—culture like fermented smell, 1-white/beige color to 10-brown/orange colour). Texture was judged by the sensation of a grainy or non-grainy feeling when rubbed onto the skin. The same scoring system was used here, where 1 was the control, i.e. no culture powder added. An increasing score number was equivalent to an increase in the coarseness feeling of the culture powder in the formula (coarseness measured on a scale of 1-10 on how smooth the base formulation feels on the skin).

### Results

Stability testing

FIG. 1 shows the viability of fine grain and standard Lactobacillus reuteri DSM 17938 culture powder in jars with desiccant (a) and without a desiccant (b), stored at 37° C. for over one month. FIG. 2 shows the water content in the composition with fine grain and standard Lactobacillus reuteri DSM 17938 culture powder in jars with and without desiccant.

The results of the stability testing (FIG. 1) show that the viability of the Lactobacillus reuteri DSM 17938 culture powder was markedly higher with desiccant (a) compared to without the desiccant (b). The water content of both fine grain and standard grain compositions was significantly lower at 1 month with the desiccant (a) compared to without the desiccant (b), which may account for the improved stability of the Lactobacillus reuteri DSM 17938 in a composition with a desiccant as shown in FIG. 1.

### Sensory Analysis

As shown in FIG. 3, after storage at 37° C., all samples had a change in smell (odor) compared to baseline. The scoring data showed that the two samples without desiccants had the biggest increase in unpleasant smell. Standard culture powder without desiccant resulted in the highest scoring, i.e. the largest change in smell, compared to all other test samples. The fine grain culture powder with the desiccant showed hardly no development of unpleasant smell from the baseline to after a 2 month storage period (FIG. 3a).

During storage at 37° C., all test samples except for the control sample also changed color over time. The samples without a desiccant showed the biggest change in color. With desiccant, the change in color was greater with the standard culture powder than with the fine grain powder (FIG. 4a).

Also, the texture of the product that resulted in the highest scoring was the standard culture powder where it, already at Day 0, was possible to feel the culture powder in the product when it was rubbed onto the skin. The fine grain culture powder could not be felt in the product when rubbed onto the skin at baseline and showed very little change in texture when stored for 2 months, which is a very positive result.

### Example 3—Lactobacillus Reuteri DSM 17938 as a Novel Topical Cosmetic Ingredient

The main aim of the study was to investigate the cutaneous acceptability of an ointment composition comprising a fine grain culture powder of Lactobacillus reuteri DSM 17938. The cutaneous acceptability was assessed by firstly, observing physical signs directly linked to the investigational product through nature, location and sensitivity. Secondly through a questionnaire filled in by each subject on the functional signs (prickling, tightening, heating sensation) linked to the product. The questionnaire was filled in at home daily by the subject detailing application frequency and number of applications as well as experienced reactions (if any).

### Materials and Methods

### Investigational Products

The probiotic product was an ointment composition consisting of a semi-solid anhydrous base formulation containing shea butter, canola oil, hydrogenated vegetable fat and a fine grain culture powder of Lactobacillus reuteri DSM 17938 (deposited to Deutsche Sammlung von Mikroorganismen and Zellkulturen GmbH, DSMZ, on 2006-01-30) as described in Example 1 and 2. The strain was added to the formulation with the aim of reaching a concentration of a minimum 1×10⁸ CFU/gram. In parallel, the probiotic product was put on stability in this formulation for the duration of the study at both 25° C. and 5° C. in order to monitor the stability of the probiotic bacteria using standardized laboratory protocols.

The control product was an identical product but without the probiotic bacteria. The odor, color and consistency of the two products were the same. Subjects were randomized to apply either probiotic product, or control product and were instructed to apply the ointment on the whole body twice per day (morning and evening), for 8 weeks.

### Patient Recruitment and Sampling

The study included 36 subjects. The subjects were aged between 18-70 years old diagnosed with AD (atopic dermatitis), diagnosed according to the definition of the U.K Working Party's Diagnostic Criteria for Atopic Dermatitis (SCORAD index of >25). Other specific inclusion criteria included subjects presenting with a current lesion of AD on a defined area.

The primary endpoint was to evaluate the cutaneous acceptability of the products, which was performed by the dermatologist investigator through a physical examination (Day 0 and Day 28), and by the subjects through completion of a questionnaire (Day 0, Day 28).

Secondary evaluation was improvement of skin appearance evaluated by the dermatologist through SCORAD and Local-SCORAD (Day 0, Day 28, Day 56), and cosmetic qualities and efficacy through questionnaires filled in by the subject in the presence of the dermatologist on Day 28.

### Appraisal of the Cutaneous Acceptability by the Dermatologist Investigator

Cutaneous acceptability of cosmetic products was assessed by the dermatologist investigator. The clinical examination was carried out by the Dermatologist investigator before the start of the study, and after 4 weeks (Day 28) of continuous application. The cutaneous acceptability was assessed by firstly, observing physical signs directly linked to the investigational product through nature, location and sensitivity. Secondly through a questionnaire filled in by each subject on the functional signs (prickling, tightening, heating sensation) linked to the product. The questionnaire was filled in at home daily by the subject detailing application frequency and number of applications as well as experienced reactions (if any).

### Cosmetic Acceptability

Cosmetic acceptability was evaluated using standardized evaluation procedures. Appraisal of the cosmetic acceptability and efficacy of the products by the participant were assessed through questionnaires adapted to the investigational products in the presence of the study monitor and was filled in on the final visit by the subject (scale: very good, good, rather good, mediocre, bad). The questionnaire is then completed by an appraisal of the products in the presence of the dermatologist.

### Clinical Evaluation by the Dermatologist Investigator

SCORing Atopic Dermatitis (SCORAD) and was evaluated by using the standardized form from the European task force on Atopic Dermatitis (Kunz B, Oranje A P, Labrèze L, Stalder J F, Ring J, Taïeb A. Clinical validation and guidelines for the SCORAD index: consensus report of the European Task Force on Atopic Dermatitis. Dermatology. 1997;195(1):10-19. doi:10.1159/000245677). Evaluation of each parameter of SCORAD which includes the sum of the total involved area, intensity of the lesions (xerosis, erythema, excoriation) and subjective symptoms (itching, sleep loss), for each subject was performed before the start of use, after 4 weeks of application and after 8 weeks of application by the Dermatologist. Local SCORAD (score of intensity of the physical signs) was evaluated by the Dermatologist investigator, at each visit, on the recurrent lesion and on the control area which was selected on Day 0.

### Results

The primary outcome of this study was to investigate the cutaneous acceptability of two topical products in adult subjects with atopic dermatitis (AD). As mentioned above, two groups were included in the study, one group received the probiotic product and the other group received the control product. The number of subjects in each group was equal and the mean age of each group was close to similar. Skin type was also similar between each group, 65% of the subjects were black in the control group while 58% were black in the probiotic group. The average volume of product used after 8 weeks was 62 g, with an overall range between 33.1 g-128.1 g from the 30 individuals who returned the products at the end of the study. Stability of the fine grain powder of Lactobacillus reuteri DSM 17938 was monitored in the product using validated methods, for the duration of the study period and was stable (data not shown).

### Cutaneous and Cosmetic Acceptability of the Investigational Products

For the probiotic containing product, the results obtained from the dermatologist investigators evaluation and questionnaires revealed a very good acceptability of the investigational product containing the probiotic product. Moderate erosion, slight erythema, erosion, edema, slight edema and dryness were identified for a few subjects. Some felt discomfort and presented with a redness or dryness of slight to moderate intensity. However, the intensity, duration and frequency of the appearance of these reactions are frequently encountered with this type of investigational product studied under normal conditions of use. For the second investigational product (the control product), the results were about the same. The subjects also concluded that the products were acceptable. The overall analysis of the reactions observed for these two investigational products, allows us to conclude that the acceptability level is equivalent for both products and are acceptable for use in adults with AD.

### Time Effect of Investigational Products (Both With and Without Live Probiotic Bacteria) on the Reduction of SCORAD and Local-SCORAD

The total number of subjects analyzed for efficacy of the two products was n=33. SCORAD and local SCORAD were calculated at baseline, day 28 (visit 2) and day 56 (end of study visit) on 17 subjects in the probiotic group and 16 subjects in the control group (one subject for day 28, and another subject for day 56).

The probiotic product was shown to reduce SCORAD (mean reduction) compared to the control (FIG. 6). These results may be further strengthened by including additional subjects in the study and treating the subjects for longer periods of time.

There was a difference in the mean reduction of local-SCORAD between the group using the probiotic and the group using the control product (FIG. 7). These results may be further strengthened by including additional subjects in the study and treating the subjects for longer periods of time. Still, within the SCORAD-Intensity of Lesions score (Sum of Erythema, Edema, Oozing, Excoriation, Lichenification and Dryness scores) a stronger reduction in dryness of the probiotic product compared to the control after 8 weeks of continuous use was observed (FIG. 8b).

A positive reduction was also seen for dryness for local-SCORAD (local-SCORAD-Intensity of Lesions score), where the probiotic product showed an improved mean variation in reducing dryness compared to the control product (FIG. 8a).

### Discussion

Atopic Dermatitis (AD) effects the quality of life both physically and emotionally of subjects who suffer from the disorder and topical emollients are extremely important in its management. This study investigated two products, one containing live probiotic bacteria, a fine grain culture powder of L. reuteri DSM 17938 and one lacking a live probiotic. The two products used on subjects with AD in this study were both concluded to be cutaneous acceptable by the dermatologist investigator. It was observed from these results that L. reuteri DSM 17938 as an additive ingredient to the topical emollient does not affect the cosmetic or cutaneous acceptability of the product. As well as this, AD is a multi-factorial condition that usually needs multiple management steps in order to manage the condition, including specific bathing instructions, clothing and avoiding allergens. In the case of topical products, it is important that the product is suitable for use on the skin, therefore it is important to understand that the control product in this case is an effective product in itself in the management of AD, as it contains other barrier protecting ingredients, like shea butter.

It was observed in this study that both products showed an improvement of the SCORAD index after 4 weeks and 8 weeks of use on the basis of evaluation by the dermatologist investigator (FIG. 7). The probiotic product showed a greater improvement in symptoms of the SCORAD after 8 weeks compared to control product. The results in Local-SCORAD after 8 weeks was similar to SCORAD for both products (FIG. 7).

In this study we saw promising results supporting that L. reuteri DSM 17938 containing emollient decreases dryness compared to product without it (FIGS. 8a and 8b). It was observed in this study that both products could significantly reduce itching and sleep loss (data not shown), and that the probiotic product showed an improvement to the control product in this regard. As mentioned, L. reuteri DSM 17938 is a widely researched and characterized probiotic strain that has been shown to have anti-inflammatory and pathogen inhibitory effects, however this is the first time, to our best knowledge that it has been combined in an topical ointment composition for use in subjects with AD.

### Conclusion

This study demonstrates that a probiotic containing ointment composition is both cutaneously acceptable and safe for use on the skin, and importantly showed a statistically and clinically significant improvement of the SCORAD index and Local SCORAD in adult subjects with AD after 4 and 8 weeks of continuous use.

The probiotic product, containing L. reuteri DSM17938 as an extra ingredient shows promise as a novel topical cosmetic emollient or ointment composition and with further testing could be a standard topical product for the management of AD or other conditions associated with the skin.

### Example 4—Long-Term Stability, Appearance and Sensory Study

A long-term stability study was performed on a product comprising an ointment composition with a formulation as follows: Lipex Sheasoft 50%, Lipex Basol C 46.5%, Dermofeel Viscolid (palm free) 2.5% and fine grain L. reuteri DSM 17938 culture powder 1% (which is the same ointment composition as described in example 1).

The product was packaged into aluminum tubes, 25 ml with a desiccant strip (45 mm×7.5 mm×1.6 mm, M-0026 Activ-film from CSP Technologies).

### Study Design

Samples were stored at each of the following conditions:
24 months in 5±3° C./ambient RH
24 months in 25±2° C./60 ±5% RH
24 months in 30 ±2° C./75 ±5% RH

The samples were (or will be) analysed at start (0 months), 3, 6, 12, 18, and 24 months.

At each time point, the activity of L. reuteri DSM 17938 was measured according to the stability testing described in Example 2. The appearance (color) testing was made according to a visual assessment, whereas the sensory (odor) testing was made by smelling the sample. For the appearance and sensory analyses, the samples stored at 5° C. were considered as a reference and were compared to the results from the samples stored at 25° C. and 30° C.

### Results

To date, samples for all time points up to 18 months have been analyzed and the results show (see table below) that samples stored at both 25° C. and 30° C. have good long-term stability capabilities as both the viability of the bacteria is high (low log loss as compared to the starting point) and the appearance (data shown below) and sensory results (data not shown) are acceptable (referred to as OK, which corresponds to a grading of 1-3 on the scale mentioned in Example 2), as compared to the 5° C. samples. The target was to have a viability of 1,0E+8 CFU/g at the end of shelf life after 24 months of storage.

TABLE 1

| Month | 0 | 3 | 6 | 12 | 18 |
|---|---|---|---|---|---|
| 5° C., amb RH | | | | | |
| L. reuteri CFU/g | 2.3E+09 | 2.3E+09 | 2.0E+09 | 1.9E+09 | 2.3E+09 |
| L. reuteri log CFU/g | 9.35 | 9.36 | 9.30 | 9.29 | 9.37 |
| Log loss from start | — | 0.00 | 0.05 | 0.07 | −0.01 |
| Appearance | OK | OK | OK | OK | OK |
| 25° C., 60% RH | | | | | |
| L. reuteri CFU/g | 2.3E+09 | 1.6E+09 | 1.1E+09 | 9.7E+08 | 9.0E+08 |
| L. reuteri log CFU/g | 9.35 | 9.19 | 9.06 | 8.99 | 8.96 |
| Log loss from start | — | 0.16 | 0.30 | 0.37 | 0.40 |
| Appearance | OK | OK | OK | OK | OK |
| 30° C., 75% RH | | | | | |
| L. reuteri CFU/g | 2.3E+09 | 1.5E+09 | 1.1E+09 | 9.0E+08 | 7.5E+08 |
| L. reuteri log CFU/g | 9.35 | 9.16 | 9.06 | 8.95 | 8.87 |
| Log loss from start | — | 0.19 | 0.30 | 0.40 | 0.48 |
| Appearance | OK | OK | OK | OK | OK |

### REFERENCES

1. U.S. Pat. No. 4,518,696

2. U.S. publ. No. 20050271641

3. WO2012038414

4. EP2364712A1

5. Kunz B, Oranje A P, Labrèze L, Stalder J F, Ring J, Taïeb A. Clinical validation and guidelines for the SCORAD index: consensus report of the European Task Force on Atopic Dermatitis. Dermatology. 1997;195(1):10-19. doi:10.1159/000245677.

1. A container comprising:
i) an ointment composition comprising an anhydrous base formulation and a fine grain culture powder of dried viable probiotic bacteria having a particle size distribution of d(90)<250 μm,
wherein said anhydrous base formulation comprises a vegetable fat, a vegetable oil and a hydrogenated vegetable oil; and
ii) a solid support comprising a desiccant polymer,
wherein said anhydrous base formulation is a solid or a semi-solid phase formulation at about 25° C.

2. The container of claim 1, wherein said vegetable fat is shea butter, said vegetable oil is canola oil and said hydrogenated vegetable oil is hydrogenated canola oil.

3. The container of claim 1 or 2, wherein said anhydrous base formulation comprises:
i) 45% to 80% by weight of vegetable fat,
ii) 20% to 50% by weight of a vegetable oil; and
iii) 0.1% to 15% by weight of a hydrogenated vegetable oil.

4. The container of claims 1 to 2, wherein said anhydrous base formulation comprises:
i) 45% to 55% by weight of a vegetable fat;
ii) 40% to 50% by weight of a vegetable oil; and
iii) 0.5% to 10% by weight of a hydrogenated vegetable oil.

5. The container of claim 4, wherein said anhydrous base formulation comprises:
i) about 50.5% by weight of a vegetable fat, such as shea butter;
ii) about 47.0% by weight of a vegetable oil, such as canola oil; and
iii) about 2.5% by weight of a hydrogenated vegetable oil, such as hydrogenated canola oil.

6. The container of any one of claims 1 to 5, wherein said solid support is selected from the group consisting of a strip, a pad, a bead or a film, or is present as a moulding in a packaging material of said container.

7. The container of any one of the preceding claims, wherein the final content of probiotic bacteria in said ointment composition is about 0.001 to 10%, such as 0.01% to 5% by dry weight, such as about 1% per dry weight of the total ointment composition.

8. The container of any one of the preceding claims, wherein said probiotic bacteria is lactic acid bacteria, optionally Lactobacillus, such as a Lactobacillus reuteri strain, such as Lactobacillus reuteri DSM 17938.

9. The container of any one of the preceding claims, wherein said container is made of aluminum, glass or plastic, cork, rubber or a combination thereof.

10. The container of any one of the preceding claims, wherein said container is air-tight.

11. An ointment composition comprising an anhydrous base formulation and a fine grain culture powder of dried viable probiotic bacteria with a particle size distribution of d(90)<250 μm,
wherein said anhydrous base formulation comprises a vegetable fat, a vegetable oil and a hydrogenated vegetable oil and wherein said anhydrous base formulation is a solid or a semi-solid phase formulation at about 25° C.

12. The composition of claim 11, wherein said vegetable fat is shea butter, said vegetable oil is canola oil and said hydrogenated vegetable oil is hydrogenated canola oil.

13. The composition of claim 11 or 12, wherein said anhydrous base formulation comprises:
i) 45% to 80% by weight of vegetable fat,
ii) 20% to 50% by weight of a vegetable oil; and
iii) 0.1% to 15% by weight of a hydrogenated vegetable oil.

14. The composition of claim 11 or 12, wherein said anhydrous base formulation comprises:
i) 45% to 55% by weight of a vegetable fat;
ii) 40% to 50% by weight of a vegetable oil; and
iii) 0.5% to 10% by weight of a hydrogenated vegetable oil.

15. The composition of claim 13 or 14, wherein said vegetable fat is shea butter, said vegetable oil is canola oil and said hydrogenated vegetable oil is hydrogenated canola oil.

16. The composition of claim 14, wherein said anhydrous base formulation comprises:
i) about 50.5% by weight of a vegetable fat, such as shea butter;
ii) about 47% by weight of a vegetable oil, such as canola oil; and
iii) about 2.5% by weight of a hydrogenated vegetable oil, such as rapeseed oil.

17. The composition of any one of claims 11 to 16, wherein the final content of probiotic bacteria is about 0.001 to 10%, such as 0.01% to 5% by dry weight, such as about 1% per dry weight of the total ointment composition.

18. The composition of any one of claims 11 to 17, wherein said probiotic bacteria are lactic acid bacteria, optionally Lactobacillus, such as a Lactobacillus reuteri strain, such as Lactobacillus reuteri DSM 17938.

19. Use of the composition of any one of claims 11 to 18, for non-therapeutic topical administration to a skin surface of a subject.

20. The composition of any one of claims 11 to 18, for use in therapeutic topical administration to a skin surface of a subject.

21. The composition of any one of claims 11 to 18, for use in a method of treating or preventing atopic dermatitis.

22. A method for therapeutic topical administration of an ointment composition of any one of claims 11 to 18, said method comprising topically applying or administering a pharmaceutically or therapeutically effective amount of said ointment composition to a skin or a mucosal surface of a subject in need thereof.

23. A method for treating or preventing skin conditions or disorders, such as atopic dermatitis, in a subject, said method comprising topically, such as to a skin or a mucosal surface, administering a pharmaceutically or therapeutically effective amount of an ointment composition of any one of claims 11 to 18 to a subject in need thereof.

24. The composition of any one of claims 11 to 18, or the composition for use of any one of claims 19 to 21, wherein said composition is provided together with a solid support comprising a desiccant polymer, optionally wherein said solid support is selected from the group consisting of a strip, a pad, a bead or a film, or is present as a moulding in a packaging material of a container for holding said composition.

25. Use of a composition of any one of claims 11 to 18 as a cosmetic, optionally wherein said composition is provided together with a solid support comprising a desiccant polymer, optionally wherein said solid support is selected from the group consisting of a strip, a pad, a bead or a film, or is present as a moulding in a packaging material of a container holding said composition.

26. A method for preparing an ointment composition of any one of claims 11 to 18, said method comprising the steps of:
i) mixing an anhydrous base formulation comprising a vegetable oil, a vegetable fat and a hydrogenated vegetable oil at about 65 to 70° C. until the fats are melted;
ii) cooling the mixture of step i) to about 30°; and
iii) mixing the anhydrous base formulation with a culture powder of dried viable probiotic bacteria to obtain a solid or a semi-solid ointment composition.

27. An ointment composition obtainable by the method of claim 26.

28. A method for prolonging the shelf life, improving the stability and/or preventing changes in the color or smell of an ointment composition comprising performing a method of claim 26, and thereafter:
iv) obtaining the solid or semi-solid ointment composition of step iii) of claim 26 and placing said composition in a container;
v) placing a solid support comprising a desiccant polymer in said container together with said ointment composition, optionally wherein said solid support is a strip, a pad, a bead or a film and/or wherein said solid support comprising a desiccant polymer is present in the packaging material of said container; and
vi) sealing said container.

29. A container comprising an ointment composition and a desiccant obtainable by the method of claim 28.

30. Use of a solid support comprising a desiccant polymer for improving the stability and/or shelf life of an ointment composition of any one of claims 11 to 18.

31. Use of a solid support comprising a desiccant polymer for preventing changes in the color and/or smell of an ointment composition of any one of claims 11 to 18.
